(19) United States
(12) Patent Application Publication
(10) Pub. No.: US 2010/0286074 A1
(43) Pub. Date: Nov. 11, 2010

(54) PHARMACEUTICAL USE OF GINSENOSIDE OR MIXTURE THEREOF AND PHARMACEUTICAL COMPOSITION OF GINSENOSIDE AND USE THEREOF

(75) Inventors: Yitao Wang, Guangdong (CN); Shaoping Li, Guangdong (CN); Hui Cao, Guangdong (CN); Zhaohua Peng, Guangdong (CN); Juhua Hu, Guangdong (CN)

Correspondence Address: CHALKER FLORES, LLP, 2711 LBJ FRWY, Suite 1036, DALLAS, TX, 75234, US

(73) Assignee: LIMIN PHARMACEUTICAL FACTORY, LIVZON GROUP, Guangdong (CN)

(21) Appl. No.: 12/746,737

(22) PCT Filed: May 12, 2008
(86) PCT No.: PCT/CN08/01981

§ 371 Date: Jul. 8, 2010

(30) Foreign Application Priority Data
Dec. 7, 2007 (JP) CN200710195591.2

(51) Int. Cl. A61K 31/704; C07J 17/00; A61P 7/02

(52) U.S. Cl. 514/26; 536/5

(57) ABSTRACT

The present invention provides a use of ginsenosides represented by formula I or mixtures thereof for manufacturing a medicament for anti-platelet aggregation, wherein R₁ and R₂ are H or glucosyl, and when R₁ is glucosyl, R₂ is H; when R₁ is H, R₂ is glucosyl. The present invention further provides a pharmaceutical composition for anti-platelet aggregation, which is capable to be used to prevent and treat thrombosis. The present invention also provides a method for generating the effect of anti-platelet aggregation in patients in vivo.

### TECHNICAL FIELD

The present invention involves a use of ginsenoside and mixture thereof in manufacturing an active medicament for anti-platelet aggregation. It also involves a pharmaceutical composition for anti-platelet aggregation and a method for generating the effect of anti-platelet aggregation in patients in vivo.

### BACKGROUND ART

Blood platelet is a kind of acaryotes produced by the megakaryocytes in bone marrow, which are the smallest particles present in blood and have complicated ultrafine structure as well as special physiological functions of adherence, aggregation and release. The major physiological function of blood platelet is to participate in normal hemostasis and thrombosis under pathological conditions in vivo.

In normal blood circulation, blood platelets flow smoothly together with other blood ingredients in vessels. However, when vascular endothelium is injured and anti-thrombus function of vascular endothelial cells decreases, blood platelets will contact with the injured endothelial, such as collagen fibers and the like exposed by the heart valves in patients of rheumatic heart disease and atherosclerotic plaque etc, thereby reactions of adhesion, aggregation and release are induced. Platelet adhesion reaction is generated by the combination of platelet membrane glycoproteins and a variety of adhesive proteins which may combine with the damaged subendothelial connective tissues, such as collagen and microfibers. Adhesive proteins include fibrinogen and fibronectin and the like, through which the activated platelet membrane glycoprotein IIb/IIIa will combine with the subendothelial tissue. Adhesive platelets will deform with pseudopodia hung out along the surface of vessels, and then are activated and aggregate together. Activated platelets will conduct a release reaction to release adenosine diphosphate (ADP), 5-hydroxy tryptamine (5-HT), adrenaline and histamine as well as other substance, which enable more platelets to accumulate more densely and form tight and deaggregated aggregates, i.e. thrombus. Thus, the drugs which can prevent platelet aggregation play an active role in anti-thrombosis.

Ginsenoside exists in ginseng, radix notoginseng and other medicinal plants extensively, which includes the monomer ginsenoside Rd, Re, Rb1, Rb2, Rc, Rg1, Rg2 and Rh1 and so on. It is generally thought that ginsenosides not only can reduce the content of free radicals in vivo, but also have the effects of anti-aging, anti-fatigue, enhancing memory, reducing wrinkles, activating skin cells, improving skin elasticity, as well as preventing tumor.

As one of the monomers, ginsenoside Rh1 has been drawn attention of many researchers. Chinese patent application titled “Pharmaceutical Use of Ginsenoside Rh1” with the application number 200510035351.7 and publication number CN1883491 published on Dec. 27, 2006, disclosed the use of ginsenoside Rh1 for preparing a medicament with the effects of improving memory, protecting nerve, anti-cerebral ischemia, stimulating hemopoietic function of bone marrow, resisting cartilage degeneration and preventing cataract. Chinese patent ZL 200410033697.9 with the publication number CN1689552 published on Nov. 2, 2005 disclosed a cosmetic of anti-skin aging, in which the use of ginsenoside Rh1 in cosmetic formula for preventing skin aging was involved.

Chinese patent application 200610008476.5 with the publication number CN1839859 published on Oct. 4, 2006 indicates the use of ginsenoside Rh1 in preparing a medicament for treating aplastic anemia; Chinese patent application 200610008477.X with the publication number CN1839860 published on Oct. 4, 2006 disclosed the use of ginsenoside F1 in preparing a medicament for treating Parkinson's disease; Chinese patent application 200610008475.0 with the publication number CN1839858 published on Oct. 4, 2006 disclosed the use of ginsenoside F1 in preparing a medicament for treating cataract; and Chinese patent application 200610008474.6 with the publication number CN1839857 published on Oct. 4, 2006 disclosed the use of ginsenoside F1 in preparing a medicament for treating ischemic heart disease and ischemic cerebrovascular disease.

### DISCLOSURE OF INVENTION

One purpose of the present invention is to provide a pharmaceutical use of ginsenoside or mixture thereof.

Another purpose of the present invention is to provide a pharmaceutical composition and its use, wherein the pharmaceutical composition comprises ginsenoside or mixture thereof and pharmaceutically acceptable excipients.

A still another purpose of the present invention is to provide a method for generating the effect of anti-platelet aggregation in patients in vivo.

In one aspect, the present invention provides the use of ginsenoside represented by the following formula I and mixture thereof for manufacturing a medicament for anti-platelet aggregation:

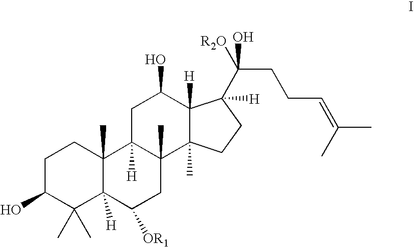

wherein, R₁ and R₂ are H or glucosyl, and when R₁ is glucosyl, R₂ is H; when R₁ is H, R₂ is glucosyl.

When R₁ is glucosyl and R₂ is H, the structure of ginsenoside of formula I is as follows:

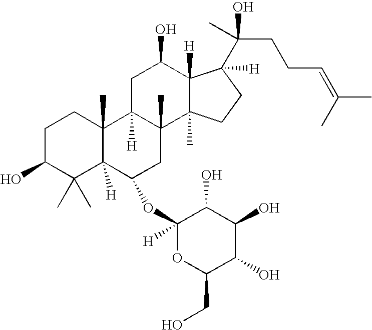

Ginsenoside as above is ginsenoside Rh1 with a molecular weight of 638.87 and CAS number of 63223-86-9.

When R₁ is H and R₂ is glucosyl, the structure of ginsenoside of formula I is as follows:

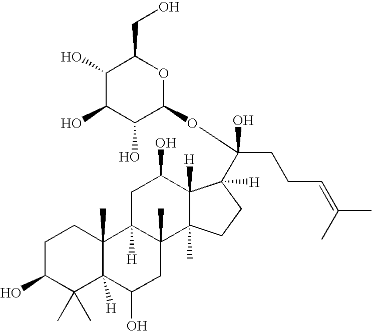

Ginsenoside as above is ginsenoside F1, which is an isomer of Rh1 with a molecular weight of 638.87.

The present inventor found surprisingly that ginsenoside Rh1 and ginsenoside F1 or their mixture all have the function of anti-platelet aggregation.

Preferably, wherein R₁ is glucosyl and R₂ is H.

Preferably, the medicament for anti-platelet aggregation is a medicament for anti-platelet aggregation induced by adenosine diphosphate.

Preferably, the medicament for anti-platelet aggregation is a medicament for preventing thrombosis.

Preferably, the medicament for anti-inhibiting platelet aggregation is a medicament for treating thrombus.

Preferably, the medicament for anti-platelet aggregation is in the dosage form of tablet, granule, dropping pill, capsule, powder injection or injection.

More preferably, the medicament for anti-platelet aggregation is in the dosage form of tablet.

In another aspect, the present invention provides a pharmaceutical composition for anti-platelet aggregation, wherein the pharmaceutical composition comprises ginsenoside represented by the following formula I or mixture thereof as well as pharmaceutically acceptable excipients,

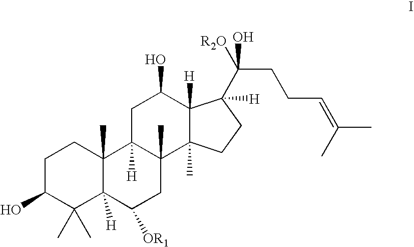

wherein, R₁ and R₂ are H or glucosyl, and when R₁ is glucosyl, R₂ is H; and when R₁ is H, R₂ is glucosyl.

Preferably, R₁ is glucosyl and R₂ is H.

In still another aspect, the present invention provides a method for generating the effect of anti-platelet aggregation in patients in vivo. This method comprises administering the pharmaceutical composition of the present invention to patient.

Preferably, the pharmaceutical composition is administered orally.

Preferably, the patient is the patient with thrombosis.

The pharmaceutically acceptable excipients used in the invention are those pharmaceutically acceptable excipients commonly used in the field of pharmaceutics. The tablet described in the present invention can be compressed tablet, coating tablet or effervescent tablet. When ginsenoside Rh1 of formula I is used in the preparation of tablets, the pharmaceutical excipients needed may be, for example, dextrin, starch, lactose, glucose, mannitol, sodium carboxymethylcellulose and surfactants which can increase the stability of drug; the pharmaceutical excipients needed for preparing granules are the same as those for tablets; the water-soluble excipients for the preparation of dropping pills may be, for example, polyethylene glycols, PEG 6000, PEG 4000, PEG 300 and soap excipient, such as sodium stearate, glycerol and gelatin, which may be used as the water-soluble excipients. The encapsulating materials for the preparation of capsules may adopt those materials commonly used in the art, and starch, glucose and so on may be used as excipients. The suitable pH adjustors and preservative agents may be selected as need for injection preparation.

Ginsenoside of formula I is widespread in ginseng and radix notoginseng, which can be obtained through separation and extraction as well as commercially available. Currently, ticlopidine hydrochloride has been widely used in treating ischemic heart-cerebrovascular diseases, but it is of inherent risks because it has significant side effects on the liver. The present invention has proved with clinical data that ginsenoside Rh1 and F1 can significantly reduce the risk of acute events happened in the patients with heart-cerebrovascular diseases and the risk of ischemic events happened in the patients with peripheral arterial diseases. Meanwhile, it has advantages of stronger efficacy of anti-platelet aggregation and fewer adverse reactions as compared to ticlopidine hydrochloride.

### BEST MODES FOR CARRYING OUT THE INVENTION

### Example 1

### Experiment of Ginsenoside Rh1 on Human with Regard to Anti-Platelet Aggregation

### 1. Patient Selection

### Conditions for Selection:

a. The patients with chronic cardiovascular and cerebrovascular diseases, and the course of disease >1 month;
b. The persons with significantly increased level of platelet aggregation induced by ADP (0.5 μmol/L>30% or 1.0 μmol/L>60%).

### Conditions for Exclusion:

a. The patients attacked by acute myocardial infarction or stroke in one month;
b. The patients taking aspirin or other anti-platelet aggregation drugs (including traditional Chinese medicine proved to have anti-platelet aggregation effect);
c. The patients with medical history of serious liver, kidney disease, or digestive tract or other organ bleeding;
d. The persons with reduced granulocytes or platelets.

### 2. Drugs

Experimental group: ginsenoside Rh1, manufactured tablets with a batch number of 051109, and each containing 305 mg of ginsenoside Rh1;
Control group: tablets of ticlopidine hydrochloride (Referred to as TCP, produced by Tianjin Xinxin Pharmaceutical Factory, with batch number 990301, and each containing 250 mg of TCP).

### 3. Methods

Experimental group: ginsenoside Rh1 305 mg per day, i.e. one tablet per day, and taking with breakfast;
Control group: TCP 250 mg per day, i.e. one tablet per day and taking with breakfast;

The course for both experimental group and control group are four weeks.

The basic drugs taken by the patients for treating their cardiovascular and cerebrovascular diseases were maintained the same in terms of dosage and usage during the period for observation, but they are forbidden to take anticoagulant drug, such as aspirin, dipyridamole or Phenylbutazone. The administration was stopped at the end of 4^{th} week and re-examination was performed to observe the change of indexes.

### 4. Observing Indexes

### 4.1 Platelet Aggregation:

Induction experiment of two different concentrations of ADP (0.5 μmol/L and 1.0 μmol/L) was performed and re-examination was conducted on the 2^{nd} and 4^{th} weekends.

### 4.2 Time for Bleeding and Clotting:

Duke Method and Test Tube Method were used for determination respectively. The same method was used for the same case. The values are compared before and after the determination, and the changing values are calculated.

### 5. Results and Discussion

### 5.1 Changes in Platelet Aggregation

After taking drugs for 2 weeks, the actual participants in the experiment of platelet aggregation testing in experimental group were 109, and those in control group were 105 cases. In follow-up study at the end of 4^{th} week, the participants in experimental group were 103, and those in control group were 94. The results are listed in Table 1.

TABLE 1
Comparison of changes in platelet aggregation rate of two
groups after drug administration ( x̅ ± s)

| | Two weeks | | Four weeks | |
|---|---|---|---|---|
| Group | 0.5 | 1.0 | 0.5 | 1.0 |
| platelet aggre- | 28.7 ± 22.5 | 35.4 ± 23.4 | 28.8 ± 23.4 | 34.3 ± 25.6 |
| gation rate of | | | | |
| the experiment | | | | |
| group (%) | | | | |
| platelet aggre- | 28.3 ± 23.8 | 35.3 ± 20.8 | 32.8 ± 23.1 | 37.2 ± 25.4 |
| gation rate of | | | | |
| the control | | | | |
| group (%) | | | | |

In Table 1, 0.5 and 1.0 represent the induction rate of 0.5 μmol/L of ADP and 1.0 μmol/L of ADP respectively. It is shown in the table that the platelet aggregation rates of two groups are similar after taking drugs for 2 weeks, but the platelet aggregation rate of the experiment group is smaller than that of the control group obviously after taking drugs for 4 weeks, so the efficacy of drug administered to experiment group is better than that to control group in terms of anti-platelet aggregation rate.

### 5.2 Change of Bleeding Time and Clotting Time

Bleeding time and clotting time of the cases in the two groups are both extended in varying degrees. Bleeding time and clotting time of the experiment group are extended from (1.72±1.20) and (7.17±2.59) min before taking drug to (1.89±1.11) and (7.83±2.55) min after taking drug; bleeding time and clotting time of the control group are extended from (1.73±1.35) and (6.99±2.44) min before taking drug to (2.29±1.40) and (8.12±2.30) min after taking drug, there are no obvious difference between the two groups.

### 5.3 Changes of Indexes in Laboratory Examination

Liver function, blood and urine routine of all the selected cases of the two groups do not change significantly before taking drugs and after taking drugs for 4 weeks, which are all within normal limits.

### 5.4 Adverse Reactions

Adverse reactions incidence in the experiment group and the control group are 6.86% and 11.87% respectively, with the latter is a little higher than the former, mainly characterized as dizziness, nausea, conjunctiva bleeding and bleeding spot on skin. These side effects can be automatically alleviated or disappear after stopping drug administration. There are no statistical differences between the two groups. But conjunctiva bleeding, purpura and bleeding spot on skin only present in the control group (incidence of 3.45%). There are also 2 cases with skin rash, hoarseness and mild dysphagia after taking drug for 2 weeks in the control group, which are relieved after stopping drug administration and undergoing anti-allergy treatment.

### 6. Analysis

TCP has been widely used in treating ischemic heart-cerebrovascular diseases in China, whereas the product administered to the experimental group has not entered clinical application yet. Clinical data have proved that the drug administered to the experimental group can significantly reduce the risk of acute events happened in patients with heart-cerebrovascular diseases and the risk of ischemic events happened in patients with peripheral arterial diseases, and meanwhile has advantages of stronger efficacy of anti-platelet aggregation and fewer adverse reactions than TCP.

1. A use of ginsenoside represented by the following formula I or mixture thereof for manufacturing a medicament for anti-platelet aggregation, 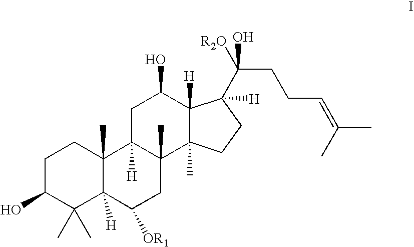
wherein, R₁ and R₂ are H or glucosyl, and when R₁ is glucosyl, R₂ is H; when R₁ is H, R₂ is glucosyl; preferably, R₁ is glucosyl, and R₂ is H.

2. The use according to claim 1, wherein the said medicament for anti-platelet aggregation is the medicament for anti-platelet aggregation induced by adenosine diphosphate.

3. The use according to claim 1, wherein the said medicament for anti-platelet aggregation is the medicament for preventing thrombosis.

4. The use according to claim 1, wherein the said medicament for anti-platelet aggregation is the medicament for treating thrombus.

5. The use according to claim 1, wherein the said medicament for anti-platelet aggregation is in the dosage form of tablet, granule, dropping pill, capsule, powder injection or injection.

6. The use according to claim 1, wherein the said medicament for anti-platelet aggregation is in the dosage form of tablet.

7. A pharmaceutical composition for anti-platelet aggregation comprising ginsenoside represented by formula I or mixture thereof and pharmaceutically acceptable excipient, 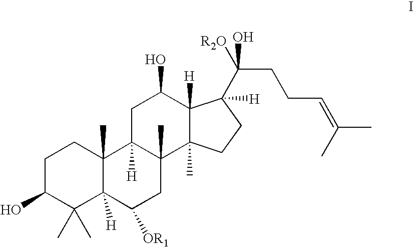
wherein R₁ and R₂ are H or glucosyl group, and when R₁ is glucosyl, R₂ is H; when R₁ is H, R₂ is glucosyl; preferably, R₁ is glucosyl group, and R₂ is H.

8. A method for generating the effect of anti-platelet aggregation in patient in vivo, which comprises administering the pharmaceutical composition according to claim 7 to patient.

9. The method according to claim 8, wherein the said pharmaceutical composition is administered orally.

10. The method according to claim 8, wherein the said patient is the patient with thrombosis.
